(19) United States
(12) Patent Application Publication
(10) Pub. No.: US 2024/0407414 A1
(43) Pub. Date: Dec. 12, 2024

(54) DIHYDROCHALCONE DERIVATIVES

(71) Applicant: SYMRISE AG, Holzminden (DE)

(72) Inventors: Yijun ZHOU, Shanghai (CN); Kai LAMOTTKE, Munich (DE); Wei JIANBING, Shanghai (CN); Johannes KIEFL, Neustadt an der Aisch (DE); Jakob Peter LEY, Holzminden (DE); Margit LIEBIG, Stadtoldendorf (DE); Bastian ZIRPEL, Holzminden (DE); Lukas SANDFORTH, Holzminden (DE)

(73) Assignee: SYMRISE AG, Holzminden (DE)

(21) Appl. No.: 18/700,293

(22) PCT Filed: Oct. 18, 2022
(86) PCT No.: PCT/EP2022/078895

§ 371 Date: Apr. 11, 2024

(30) Foreign Application Priority Data
Oct. 18, 2021 (WO) PCT/CN2021/124295

(51) Int. Cl. A23L 27/00; A23F 3/40; A23G 3/42; A23G 4/10; A23L 2/56; A23L 2/60; A23L 27/20; A23L 27/30; A23L 27/60; A61K 8/60; A61Q 11/00; C12P 19/46

(52) U.S. Cl. CPC A23L 27/88; A23F 3/405; A23G 3/42; A23G 4/10; A23L 2/56; A23L 2/60; A23L 27/2052; A23L 27/33; A23L 27/63; A23L 27/84; A23L 27/86; A61K 8/602; A61Q 11/00; C12P 19/46; A61K 2800/85; A61K 2800/92

(57) ABSTRACT

The present invention relates to the use of a compound or a mixture of two or more compounds of formula for modulating and/or optimizing the flavor of one or more sweet tasting substance(s), to a composition comprising such a compound or mixture and one or more sweet tasting substance(s) and to a product comprising such a composition. Furthermore, the present invention relates to a method for modulating and/or optimizing the flavor of one or more sweet tasting substance(s), to a method for producing a compound according to the invention and to the use of such a compound as a flavour.

The present invention relates to a compound or mixture of compounds of formula (I) as defined below for modulating and/or optimizing the flavor of one or more sweet tasting substance(s), in particular for increasing the sweet flavor and/or increasing the mouthfeel and/or reducing the acidity and/or reducing a bitter aftertaste or off-taste of one or more sweet tasting substance(s). The invention also provides a composition comprising a compound or mixture of compounds of formula (I), one or more sweet tasting substances and, optionally a sweet taste modulating substance as well as a product comprising the composition. Further, a method for modulating and/or optimizing the flavor of one or more sweet tasting substance(s), in particular for increasing the sweet flavor and/or increasing the mouthfeel and/or reducing the acidity and/or reducing a bitter aftertaste or off-taste of one or more sweet tasting substance(s) is provided. In addition, the present invention also relates to a method of producing a compound or mixture of compounds of formula (I) and finally, the present invention also provides certain new compounds or compound mixtures as well as the use of compound (1) as defined below as a flavor, in particular as sweet tasting and/or sweet modulating substance.

The sweet taste of food and beverages containing considerable quantities of sugar is often perceived as desirable by customers. However, there is a growing awareness that sugar, such as sucrose, glucose and fructose when consumed in significant quantities has a detrimental effect on customer's health. Weight gain and associated cardio-vascular issues, insulin resistance and type 2 diabetes as well as oral health problems are among the potential negative impacts of a high sugar diet.

Sweet modulating taste solutions are an important tool to reduce the sugar content in sweetened foods and beverages while maintaining the overall taste profile. Existing solutions used in the flavor industry, such as advantame, neohesperidine dihydrochalcone, rebaudiosides, rubusosides, mogrosides and erythritol come with their own disadvantages. They may have a lingering (bitter) aftertaste or off-taste and at the same time only provide low sweet modulating potency. Moreover, they are often artificial compounds, which are largely considered less acceptable in food and drinks than natural, i.e. plant based, ingredients. For this reason, there is an ongoing search for effective sweet modulating compounds with sugar-like taste of natural origin.

Stevia rebaudiana based extracts and isolates are widely used among Rubus suavissimus and Siraitia grosvenorii to improve sweet taste. Such natural sweet modulating systems are preferred solutions for product developers nowadays. However, sweet modulating terpeneglycosides such as rebaudiosides, rubusosides and mogrosides possess inherent non-sugar like taste attributes such as lingering aftertaste, licorice taste and bitterness. Dihydrochalcones are another class of sweet compounds such as neohesperidine dihydrochalcone which is, however, an artificial compound.

Trilobatin represents one of the few natural dihydrochalcones (i.e. identified in natural source material) with a reported use as sweet modulating compound (WO2008148239). However, the taste threshold is rather high and therefore a commercial use is very limited. According to WO2008148239, 20 mg/kg trilobatin does not taste sweet, sweetness can be detected above 60 mg/kg trilobatin and 100 mg/kg is only“weakly sweet” compared to 0.5% sucrose solution.

Trilobatin has been described as “sweet constituent” by He et al. among phloridizin, 3′-O-acetylphloridzin, 2′-O-acetylphloridzin and phloretin in Lithocarpus litseifolius and poly-stachyrus leaves (He et al., China Journal of Chinese Materia Medica, 37 (7), 2012). He et al. developed a method to quantify these Lithocarpus leaf constituents by UPLC-UV analysis but did not disclose any information on the sensory properties of the single or isolated compounds.

6″-O-Acetyltrilobatin and 3″-O-acetylphloridizin have been isolated from Lithocarpus litseifolius leaves and structurally elucidated for the first time by Chen et al. (Chen et al., Journal of Asian Natural Products Research, 11 (6), 2009). The numeration of carbon atoms is as follows (according to Chen et al.):

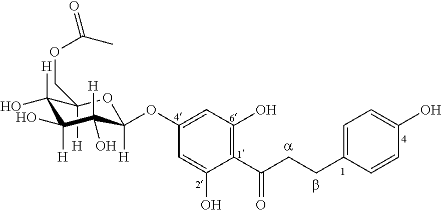

For example, phloretin-4′-β-D-glucopyranoside can be synonymously used for trilobatin. Chen et al. established the identification of 6″-O-acetyltrilobatin and 3″-O-acetylphloridizin in nature but did not disclose any sensory related information of these compounds.

2″-O-Acetyltriolbatin has been isolated from the leaves of Lithocarpus pachyphyllus by Qin X-D. and Liu J-K., Zeitschrift für Naturforschung 58c, 2003. The authors established the identification of 2″-O-acetytrilobatin and claimed this compound to be a “new sweet dihydrochalcone glucoside”, but the authors did not disclose any sensory experiments and data.

6″-O-Acetylphloridzin, on the other hand, did not show any sweet taste or sweetness enhancing properties. It can therefore not be concluded that any dihydrochalcone glycoside carrying an acetyl substituent will taste sweet or be able to enhance or modulate sweetness.

It was objective of the present invention to provide compounds with effective sweet modulating activity, in particular compounds, which are capable of increasing the sweet flavor of one or more other sweet tasting substance(s) including sweeteners and thus facilitate a reduced amount of the sweet tasting substance or sweetener in a food or beverage while maintaining the overall sweetness. In addition, it was an objective of the present invention to provide compounds, which are able to generally improve the taste of sweet tasting applications by modulating other sensory descriptors such as reducing acidity, decreasing bitterness or increasing mouthfeel. And finally, the compounds should also be of natural, i.e. plant based origin.

In the context of the present invention, it was surprisingly found out that alkanoylation of phloretin-4′-monoglucoside increases its sweet modulating property and alkanoylation can be done by enzymatic means starting with natural plant extracts.

The above mentioned objectives are met by the use of a compound or a mixture of two or more compounds of formula (I)

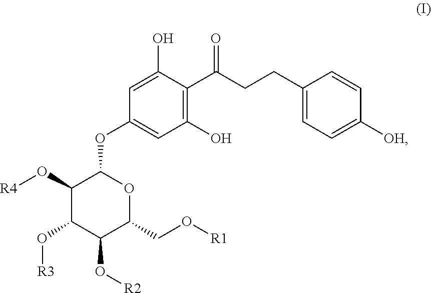

wherein R1, R2, R3 and R4 are defined as shown in the following table:

| Compound | R1 | R2 | R3 | R4 |
|---|---|---|---|---|
| (1) | acetyl | H | H | H |
| (2) | H | H | acetyl | H |
| (3) | propionyl | H | H | H |
| (4) | H | H | propionyl | H |
| (5) | acetyl | H | acetyl | H |
| (6) | H | acetyl | H | H |
| (7) | H | H | H | acetyl |
| (8) | H | propionyl | H | H |
| (9) | H | H | H | propionyl |
| (10) | butanoyl | H | H | H |
| (11) | H | butanoyl | H | H |
| (12) | H | H | butanoyl | H |
| (13) | H | H | H | butanoyl |
| (14) | acetyl | acetyl | H | H |
| (15) | acetyl | H | H | acetyl |
| (16) | H | acetyl | acetyl | H |
| (17) | H | acetyl | H | acetyl |
| (18) | H | H | acetyl | acetyl |
| (19) | acetyl | acetyl | acetyl | H |
| (20) | H | acetyl | acetyl | acetyl |
| (21) | acetyl | H | acetyl | acetyl |
| (22) | acetyl | acetyl | H | acetyl |
| (23) | acetyl | acetyl | acetyl | acetyl |
| (24) | propionyl | propionyl | H | H |
| (25) | propionyl | H | propionyl | H |
| (26) | propionyl | H | H | propionyl |
| (27) | H | propionyl | propionyl | H |
| (28) | H | propionyl | H | propionyl |
| (29) | H | H | propionyl | propionyl |
| (30) | H | propionyl | propionyl | propionyl |
| (31) | propionyl | H | propionyl | propionyl |
| (32) | propionyl | propionyl | H | propionyl |
| (33) | propionyl | propionyl | propionyl | H |
| (34) | propionyl | propionyl | propionyl | propionyl |
| (35) | butanoyl | butanoyl | H | H |
| (36) | butanoyl | H | butanoyl | H |
| (37) | butanoyl | H | H | butanoyl |
| (38) | H | butanoyl | butanoyl | H |
| (39) | H | butanoyl | H | butanoyl |
| (40) | H | H | butanoyl | butanoyl |
| (41) | butanoyl | butanoyl | butanoyl | H |
| (42) | butanoyl | butanoyl | H | butanoyl |
| (43) | butanoyl | H | butanoyl | butanoyl |
| (44) | H | butanoyl | butanoyl | butanoyl |
| (45) | butanoyl | butanoyl | butanoyl | butanoyl |

for modulating and/or optimizing the flavor of one or more sweet tasting substance(s), in particular for increasing the sweet flavor and/or increasing the mouthfeel and/or reducing the acidity and/or reducing a bitter aftertaste or off-taste of one or more sweet tasting substance(s).

Particularly preferred in the context of the present invention is the compound (1) as defined above.

Sweet tasting substances include any substance, which has a sweet taste, in particular sweeteners, but also substances, which do not provide a strong enough sweet taste on their own to qualify as sweeteners.

The compounds of formula (I) as defined above are surprisingly able modulate the sweet taste of a sweet tasting substance in a favorable way, which provides significantly increased sweetness and/or mouthfeel, reduced acidity and/or a reduced bitter aftertaste or off-taste that commonly used sweeteners exhibit (see Example 10 below). As demonstrated in Example 1 below, alkanoylated trilobatins of formula (I) have a significantly larger sensory impact than trilobatin itself. Using the same dosage, the taste of a 5% sucrose solution was perceived as 27.5% sweeter with the addition of a compound of formula (I) while trilobatin did not have a significant sensory effect. Alkanoylation therefore appears to increase the sweet modulatory properties for trilobatin. Advantageously, the compounds of formula (I) can be derived in sufficient quantities by chemical or enzymatic alkanoylation of plant extracts and thus from natural sources.

In one embodiment of the use described above, the compound or mixture of two or more compounds of formula (I) is used for modulating and/or optimizing the flavor of one or more sweet tasting substance(s) selected from the group consisting of
natural sweet tasting substance(s), preferably naturally occurring sweet tasting substances, including plant extracts, such as sweet tasting carbohydrates (such as sucrose, D-(+)-trehalose, lactose, maltose, D-(+)-melizitose, melibiose, D-(+)-raffinose, palatinose, lactulose, D-fructose, D-(+)-allulose, D-glucose, D-galactose, D-(+)-allose, D-(+)-atrose, D-(−)-gulose, D-(+)-mannose, D-(−)-idose, D-(+)-talose, L-(+)-rhamnose, L-(−)-fucose, D-sorbose, D-mannose, D-(−)-tagatose, D-arabinose, L-arabinose, D-(+)-xylose, D-lyxose, D-ribose, D-ribulose, D-(−)-erythrose, D-(+)-threose, D-glyceraldehyde, maltodextrin), sugar alcohols (such as erythritol, threitol, arabitol, ribitol, xylitol, sorbitol, mannitol, maltitol, iso-maltit, dulcitol, lactitol), proteins (such as miraculin, pentaidin, monellin, thaumatin, curculin, brazzein, mabinlin), D-amino acids (such as D-phenylalanine, D-tryptophan) or extracts or fractions obtained from natural sources containing these amino acids and/or proteins and the physiologically acceptable salts of these amino acids and/or proteins, particularly the sodium, potassium, calcium or ammonium salts thereof, steviolgylcoside, stevioside, mono-, di-, tri- or tetra-alpha-glycosylated steviosides or rebaudiosides, steviolbiosid, rebaudiosides, rebaudioside A, rebaudioside B, rebaudioside C, rebaudioside D, rebaudioside E, rebaudioside F, rebaudioside G, rebaudioside H, rebaudioside M, rebaudioside N, rebaudioside X, dulcoside, rubusoside, mono-, di-, tri- or tetra-alpha-glycosylated rubusosides, suavioside A, suavioside B, suavioside G, suavioside H, suavioside I, suavioside J, baiyunoside 1, baiyunoside 2, phlomisoside 1, phlomisoside 2, phlomisoside 3, phlomisoside 4, abrusoside A, abrusoside B, abrusoside C, abrusoside D, cyclocaryoside A cyclocaryoside I, oslandin, polypodoside A, strogin 1, strogin, 2, strogin 4, selligueanin A, dihydroquercetin-3-acetate, perillartin, telosmosid A15, periandrin I-V, pterocaryoside, cyclocaryoside, mukurozioside, trans-anethol, bryoside, bryonoside, bryonodulcoside, carnosifloside, scandenoside, gypenoside, hematoxylin, cyanin, chlorogenic acid, albiziasaponin, telosmoside, gaudichaudiosid, balansin A, balansin B, mogrosides, such as mogroside V, hernandulcine, monatin, glycyrrhetinic acid and its derivatives, particularly glycyrrhizin, preferably glycyrrhizin ammonium salt; extracts or enriched fractions of such extracts such as extracts of Thaumatococcus or Stevia ssp., particularly Stevia rebaudiana, stevia leaf extract, swingle extracts, particularly Momordica or Siratia grosvenorii or Luo-Han-Guo, extracts of Glycerrhyzia ssp., particularly Glycerrhyzia glabra or Glycerrhyzia uralensis, extracts of Rubus ssp., particularly Rubus suavissimus or Rubus chingii comprising rubusoside, extracts of Mycetia balansae, preferably comprising balansin A and/or balansin B and,
synthetic sweet tasting substances, preferably selected from the group consisting of magap, sodium cyclamate or other physiologically acceptable salts of cyclamic acid, acesulfam K, saccharin, saccharin sodium salt, aspartam, superaspartam, neotam, alitam, advantam, perillartin, sucralose, lugduname, carrelame, sucrononate and sucrooctate, or mixtures thereof.

In a preferred embodiment of the use described above, the compound or mixture of two or more compounds of formula (I) is used for modulating and/or optimizing the flavor of one or more sweet tasting substance(s) described above and additionally combined with one or more sweet taste modulating substances selected from the group consisting of hesperetin, hesperetin dihydrochalcone, naringenin, phloretin, eriodictyol, homoeriodictyol, phyllodulcin, neohesperidindihydrochalkon, naringindihydrochalkon, phloretin, extracts of Hydrangea macrophylla ssp. serrata, in particular var. Oamacha, Amacha or Amagi amacha comprising active amounts of phyllodulcin.

Particularly preferably, the compound or mixture of two or more compounds of formula (I) is used for modulating and/or optimizing the flavor of one or more sweet tasting substance(s) selected from the group consisting of sucrose, fructose, glucose, steviosides, rebaudiosides, rebaudioside A, rebaudioside M, mono-, di-, tri- or tetra-alpha-glycosylated steviosides or rebaudiosides, rubusoside, mono-, di-, tri- or tetra-alpha-glycosylated rubusosides.

Further preferably, compound (1) is used for modulating and/or optimizing the flavor of one or more sweet tasting substance(s) selected from the group consisting of sucrose, fructose, glucose, steviosides, rebaudiosides, rebaudioside A, rebaudioside M, mono-, di-, tri- or tetra-alpha-glycosylated steviosides or rebaudiosides, rubusoside, mono-, di-, tri- or tetra-alpha-glycosylated rubusosides.

In another preferred embodiment of the use described above, compound (1) is used for modulating and/or optimizing the flavor of one or more sweet tasting substance(s) described above and additionally combined with one or more sweet taste modulating substances selected from the group consisting of hesperetin, hesperetin dihydrochalcone, naringenin, phloretin, eriodictyol, homoeriodictyol, phyllodulcin, neohesperidindihydrochalkon, naringindihydrochalkon, phloretin, extracts of Hydrangea macrophylla ssp. serrata, in particular var. Oamacha, Amacha or Amagi amacha comprising active amounts of phyllodulcin.

The compounds of formula (I) can be used to modulate and optimize the flavor of a range of natural and synthetic sweet tasting substances, in particular sweeteners and can therefore be applied in a variety of common or dietetic foods and beverages.

Preferably, the compound or mixture of two or more compounds of formula (I) is used in an amount, in which it is perceived as sweet or less sweet than a 1.5% sucrose solution in water. Larger amounts of the compound(s) of formula (I) are not required to achieve the desired modulatory effect and would likely not be economically reasonable. In case of compound (1) defined below, a sweetness of 1.5% sucrose solution in water is achieved at a concentration around 65 mg/kg. Thus in a preferred embodiment of the use described above, compound (1) is used in a concentration of 65 mg/kg or less with respect to the total sweetened composition or product.

Preferred is a use as described above, wherein the compound or compounds of formula (I) is/are selected from the group consisting of

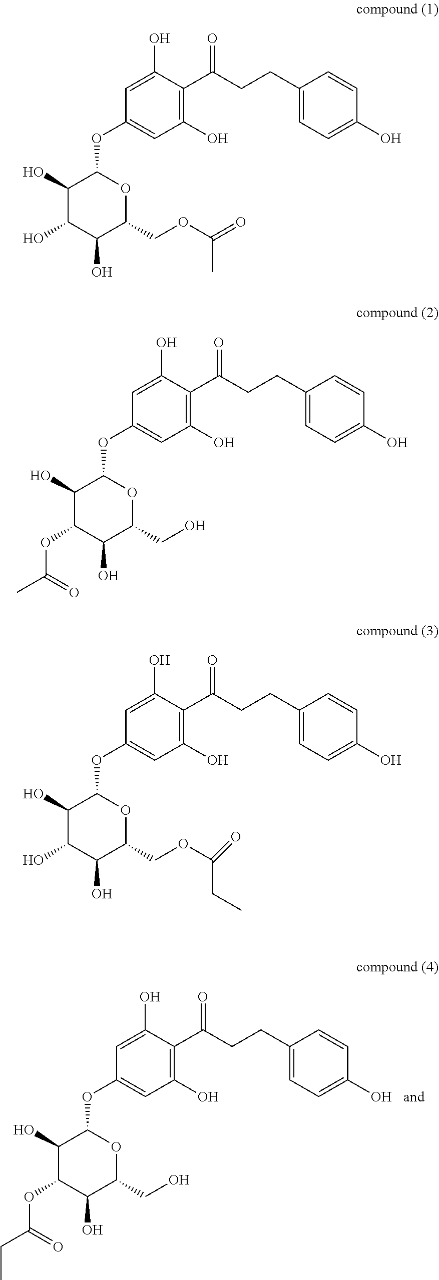 [SMILES: CC(=O)OC[C@H]1O[C@@H](Oc2cc(O)c(C(=O)CCc3ccc(O)cc3)c(O)c2)[C@H](O)[C@@H](O)[C@@H]1O ; CC(=O)O[C@@H]1[C@@H](O)[C@H](Oc2cc(O)c(C(=O)CCc3ccc(O)cc3)c(O)c2)O[C@H](CO)[C@H]1O ; CCC(=O)OC[C@H]1O[C@@H](Oc2cc(O)c(C(=O)CCc3ccc(O)cc3)c(O)c2)[C@H](O)[C@@H](O)[C@@H]1O ; CCC(=O)O[C@@H]1[C@@H](O)[C@H](Oc2cc(O)c(C(=O)CCc3ccc(O)cc3)c(O)c2)O[C@H](CO)[C@H]1O ; "and"]

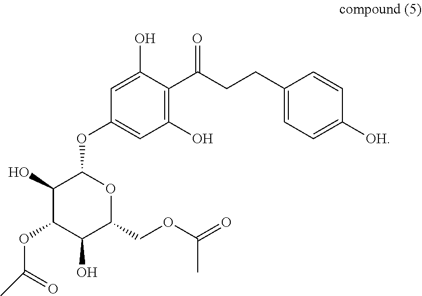 [SMILES: CC(=O)OC[C@H]1O[C@@H](Oc2cc(O)c(C(=O)CCc3ccc(O)cc3)c(O)c2)[C@H](O)[C@@H](OC(C)=O)[C@@H]1O]

Particularly preferred in the context of the present invention is compound (1).

The present invention also relates to a composition comprising or consisting of
a compound or mixture of two or more compounds of formula (I), preferably wherein the compound is obtainable or obtained by enzymatic alkanoylation of a plant extract,
and one or more sweet tasting substance(s), preferably (a) sweet tasting substance(s) selected from the group consisting of
   natural sweet tasting substance(s), preferably naturally occurring sweet tasting substances, including plant extracts, such as sweet tasting carbohydrates (such as sucrose, D-(+)-trehalose, lactose, maltose, D-(+)-melizitose, melibiose, D-(+)-raffinose, palatinose, lactulose, D-fructose, D-(+)-allulose, D-glucose, D-galactose, D-(+)-allose, D-(+)-altrose, D-(−)-gulose, D-(+)-mannose, D-(−)-idose, D-(+)-talose, L-(+)-rhamnose, L-(−)-fucose, D-sorbose, D-mannose, D-(−)-tagatose, D-arabinose, L-arabinose, D-(+)-xylose, D-lyxose, D-ribose, D-ribulose, D-(−)-erythrose, D-(+)-threose, D-glyceraldehyde, maltodextrin), sugar alcohols (such as erythritol, threitol, arabitol, ribitol, xylitol, sorbitol, mannitol, maltitol, isomaltit, dulcitol, lactitol), proteins (such as miraculin, pentaidin, monellin, thaumatin, curculin, brazzein, mabinlin), D-amino acids (such as D-phenylalanine, D-tryptophan) or extracts or fractions obtained from natural sources containing these amino acids and/or proteins and the physiologically acceptable salts of these amino acids and/or proteins, particularly the sodium, potassium, calcium or ammonium salts thereof, steviolgylcoside, stevioside, mono-, di-, tri- or tetra-alpha-glycosylated steviosides or rebaudiosides, steviolbiosid, rebaudiosides, rebaudioside A rebaudioside B, rebaudioside C, rebaudioside D, rebaudioside E, rebaudioside F, rebaudioside G, rebaudioside H, rebaudioside M, rebaudioside N, rebaudioside X, dulcoside, rubusoside, mono-, di-, tri- or tetra-alpha-glycosylated rubusosides, suavioside A, suavioside B, suavioside G, suavioside H, suavioside I, suavioside J, baiyunoside 1, baiyunoside 2, phlomisoside 1, phlomisoside 2, phlomisoside 3, phlomisoside 4, abrusoside A, abrusoside B, abrusoside C, abrusoside D, cyclocaryoside A, cyclocaryoside I, oslandin, polypodoside A, strogin 1, strogin, 2, strogin 4, selligueanin A, dihydroquercetin-3-acetate, perillartin, telosmosid A15, periandrin I-V, pterocaryoside, cyclocaryoside, mukurozioside, trans-anethol, bryoside, bryonoside, bryonodulcoside, camosifloside, scandenoside, gypenoside, hematoxylin, cyanin, chlorogenic acid, albiziasaponin, telosmoside, gaudichaudiosid, balansin A, balansin B, mogrosides, such as mogroside V, hernandulcine, monatin, glycyrrhetinic acid and its derivatives, particularly glycyrrhizin, preferably glycyrrhizin ammonium salt; extracts or enriched fractions of such extracts such as extracts of Thaumatococcus or Stevia ssp., particularly Stevia rebaudiana, stevia leaf extract, swingle extracts, particularly Momordica or Siratia grosvenorii or Luo-Han-Guo, extracts of Glycerrhyzia ssp., particularly Glycerrhyzia glabra or Glycerrhyzia uralensis, extracts of Rubus ssp., particularly Rubus suavissimus or Rubus chingii comprising rubusoside, extracts of Mycetia balansae, preferably comprising balansin A and/or balansin B and,
synthetic sweet tasting substances, preferably selected from the group consisting of magap, sodium cyclamate or other physiologically acceptable salts of cycdamic acid, acesulfam K, saccharin, saccharin sodium salt, aspartam, superaspartam, neotam, alitam, advantam, perillartin, sucralose, lugduname, carrelame, sucrononate and sucrooctate,
or mixtures thereof.

It is preferred that the composition according to the invention is obtainable or obtained by a method according to the invention.

Particularly preferably, in the composition described above, the one or more sweet tasting substance(s) is/are selected from the group consisting of sucrose, fructose, glucose, stevisodes, rebaudiosides, rebaudioside A, rebaudioside M, mono-, di-, tri- or tetra-alpha-glycosylated steviosides or rebaudiosides, rubusoside, mono-, di-, tri- or tetra-alpha-glycosylated rubusosides.

In a preferred embodiment of the composition described above, the composition additionally comprises one or more sweet taste modulating substances selected from the group consisting of hesperetin, hesperetin dihydrochalcone, naringenin, phloretin, eriodictyol, homoeriodictyol, phyllodulcin, neohesperidindihydrochalkon, naringindihydrochalkon, phloretin, extracts of Hydrangea macrophylla ssp. serrata, in particular var. Oamacha, Amacha or Amagi amacha comprising active amounts of phyllodulcin.

Particularly preferably, in the composition according to any embodiment described above, the one or more sweet tasting substance(s) is/are selected from the group consisting of sucrose, fructose, glucose, steviosides, rebaudiosides, rebaudioside A, rebaudioside M, mono-, di-, tri- or tetra-alpha-glycosylated steviosides or rebaudiosides, rubusoside, mono-, di-, tri- or tetra-alpha-glycosylated rubusosides and the composition additionally comprises one or more sweet taste modulating substance(s) selected from the group consisting of hesperetin, hesperetin dihydrochalcone, naringenin, phloretin, eriodictyol, homoeriodictyol, phyllodulcin, neohesperidindihydrochalkon, naringindihydrochalkon, phloretin, extracts of Hydrangea macrophylla ssp. serrata, in particular var. Oamacha, Amacha or Amagi amacha comprising active amounts of phyllodulcin.

Further preferably, the composition according to any embodiment described above comprises compound (1) and one or more sweet tasting substance(s) selected from the group consisting of sucrose, fructose, glucose, steviosides, rebaudiosides, rebaudioside A, rebaudioside M, mono-, di-, tri- or tetra-alpha-glycosylated steviosides or rebaudiosides, rubusoside, mono-, di-, tri- or tetra-alpha-glycosylated rubusosides.

Further preferably is a composition comprising compound (1) and one or more sweet tasting substances selected from the group consisting of sucrose, fructose, glucose, stevisodes, rebaudiosides, rebaudioside A, rebaudioside M, mono-, di-, tri- or tetra-alpha-glycosylated steviosides or rebaudiosides, rubusoside, mono-, di-, tri- or tetra-alpha-glycosylated rubusosides and one or more sweet taste modulating substance(s) selected from the group consisting of phyllodulcin, hersperetin, hesperetindihydrochalkon and phloretin or extracts of Hydrangea macrophylla ssp. serrata, in particular var. Oamacha, Amacha or Amagi amacha comprising active amounts of phyllodulcin.

The present invention further relates to a product comprising the composition as described above, wherein the amount of the compound or mixture of two or more compounds of formula (I) is less than 150 ppm, preferably less than 100 ppm, particularly preferably less than 65 ppm with respect to the total product.

The product can be selected from the group consisting of pharmaceutical products for oral application, oral care products, liquid and solid products for nutrition or pleasure.

The present invention also provides semi-finished products, which comprise 0.1 to 0.2 wt.-% of the compound or mixture of two or more compounds of formula (I). Semi-finished products are often aroma compositions, which are added to a product before use. Such semi-finished products may be free of the sweet tasting substances as defined above, but may contain the sweet modulating substances as defend above.

A semi-finished product according to the invention therefore comprises a compound or mixture of two or more compounds of formula (I), in particular compound (1), and one or more sweet taste modulating substances selected from the group consisting of phyllodulcin, hersperetin, hesperetindihydrochalkon and phloretin or extracts of Hydrangea macrophylla ssp. serrata, in particular var. Oamacha, Amacha or Amagi amacha comprising active amounts of phyllodulcin.

Preferably, the product may be selected from the group consisting of (reduced-calorie) baked goods (e.g. bread, dry biscuits, cakes, other baked articles), confectionary (e.g. muesli bar products, chocolates, chocolate bars, other products in bar form, fruit gums, dragées, hard and soft caramels, chewing gum), non-alcoholic drinks (e.g. cocoa, coffee, green tea, black tea, (green, black) tea drinks enriched with (green, black) tea extracts, rooibos tea, other herbal teas, fruit-containing soft drinks, isotonic drinks, refreshing drinks, nectars, fruit and vegetable juices, fruit or vegetable juice preparations), instant drinks (e.g. instant cocoa drinks, instant tea drinks, instant coffee drinks), meat products (e.g. ham, fresh sausage or raw sausage preparations, spiced or marinated fresh or salt meat products), eggs or egg products (dried egg, egg white, egg yolk), cereal products (e.g. breakfast cereals, muesli bars, precooked ready-to-eat rice products), dairy products (e.g. full-fat or reduced-fat or fat-free milk drinks, rice pudding, yoghurt, kefir, cream cheese, soft cheese, hard cheese, dried milk powder, whey, butter, buttermilk, ice-cream, partially or completely hydrolysed milk-protein-containing products), products made from soy protein or other soybean fractions (e.g. soy milk and products produced therefrom, drinks containing isolated or enzymatically treated soy protein, drinks containing soy flour, preparations containing soy lecithin, fermented products such as tofu or tempeh or products produced therefrom and mixtures with fruit preparations and optionally flavours), dairy-like preparations (milk-type, yoghurt-type, dessert-type, ice cream) from protein rich plant materials (e.g. from seed materials of oat, almond, pea, lupine, lentils, faba beans, chickpea, rice, canola), plant protein-enriched non-dairy drinks, fruit preparations (e.g. jams, sorbets, fruit sauces, fruit fillings), vegetable preparations (e.g. ketchup, sauces, dried vegetables, frozen vegetables, precooked vegetables, boiled-down vegetables), snacks (e.g. baked or fried potato crisps or potato dough products, maize- or groundnut-based extrudates), fat- and oil-based products or emulsions thereof (e.g. mayonnaise, remoulade, dressings, in each case full-fat or reduced-fat), other ready-made dishes and soups (e.g. dried soups, instant soups, precooked soups), spices, spice mixtures and in particular seasonings which are used, for example, in the snacks field, sweetener preparations, tablets or sachets, other preparations for sweetening or whitening drinks.

The product can be a dietary supplement or pharmaceutical product in the form of capsules, tablets (uncoated and coated tablets, e.g. gastro-resistant coatings), sugar-coated pills, granulates, pellets, solid mixtures, dispersions in liquid phases, as emulsions, as powders, as solutions, as pastes or as other formulations that can be swallowed or chewed.

Oral care products are formulations commonly used by those skilled in the art for cleaning and caring for the oral cavity and pharynx and for freshening the breath. Known and common oral care products are in the form of creams, gels, pastes, foams, emulsions, suspensions, aerosols, sprays as well as capsules, granules, pastilles, tablets, sweets or chewing gums, without this list of dosage forms being limiting with regard to the possible applications. Such formulations are used to clean and care for the tooth structure and oral cavity and to freshen the breath. In particular, oral care products according to the invention are preferably selected from the group consisting of toothpastes, tooth gels, mouthwashes, mouth rinses, liquids for gargling, oral or pharyngeal sprays (pump or aerosol spray), lozenges, lozenges, candies, chewing gums, chewy candies and dental care chewing gums.

The present invention also relates to a method for modulating and/or optimizing the flavor of one or more sweet tasting substance(s), in particular for increasing the sweet flavor and/or increasing the mouthfeel and/or reducing the acidity and/or reducing a bitter aftertaste or off-taste of one or more sweet tasting substance(s), comprising the steps:
(i) providing a compound or mixture of two or more compounds of formula (I),
(ii) combining the compound or mixture of two or more compounds of formula (I) from step (i) with one or more sweet tasting substance(s), preferably (a) sweet tasting substance(s) selected from the group consisting of
natural sweet tasting substance(s), preferably naturally occurring sweet tasting substances, including plant extracts, such as sweet tasting carbohydrates (such as sucrose, D-(+)-trehalose, lactose, maltose, D-(+)-melzitose, melibiose, D-(+)-raffinose, palatinose, lactulose, D-fructose, D-(+)-allulose, D-glucose, D-galactose, D-(+)-allose, D-(+)-atrose, D-(−)-gulose, D-(+)-mannose, D-(−)-idose, D-(+)-talose, L-(+)-rhamnose, L-(−)-fucose, D-sorbose, D-mannose, D-(−)-tagatose, D-arabinose, L-arabinose, D-(+)-xylose, D-lyxose, D-ribose, D-ribulose, D-(−)-erythrose, D-(+)-threose, D-glyceraldehyde, maltodextrin), sugar alcohols (such as erythritol, threitol, arabitol, ribitol, xylitol, sorbitol, mannitol, maltitol, iso-maltit, dulcitol, lactitol), proteins (such as miraculin, pentaidin, monellin, thaumatin, curculin, brazzein, mabinlin), D-amino acids (such as D-phenylalanine, D-tryptophan) or extracts or fractions obtained from natural sources containing these amino acids and/or proteins and the physiologically acceptable salts of these amino acids and/or proteins, particularly the sodium, potassium, calcium or ammonium salts thereof, steviolgylcoside, stevioside, mono-, di-, tri- or tetra-alpha-glycosylated steviosides or rebaudiosides, steviolbiosid, rebaudiosides, rebaudioside A, rebaudioside B, rebaudioside C, rebaudioside D, rebaudioside E, rebaudioside F, rebaudioside G, rebaudioside H, rebaudioside M, rebaudioside N, rebaudioside X, dulcoside, rubusoside, mono-, di-, tri- or tetra-alpha-glycosylated rubusosides, suavioside A, suavioside B, suavioside G, suavioside H, suavioside I, suavioside J, baiyunoside 1, baiyunoside 2, phlomisoside 1, phlomisoside 2, phlomisoside 3, phlomisoside 4, abrusoside A, abrusoside B, abrusoside C, abrusoside D, cyclocaryoside A cycocaryoside I, oslandin, polypodoside A, strogin 1, strogin, 2, strogin 4, selligueanin A, dihydroquercetin-3-acetate, perillartin, telosmosid A15, periandrin I-V, pterocaryoside, cycocaryoside, mukurozioside, trans-anethol, bryoside, bryonoside, bryonodulcoside, carnosifloside, scandenoside, gypenoside, hematoxylin, cyanin, chlorogenic acid, albiziasaponin, telosmoside, gaudichaudiosid, balansin A, balansin B, mogrosides, such as mogroside V, hernandulcine, monatin, glycyrrhetinic acid and its derivatives, particularly glycyrrhizin, preferably glycyrrhizin ammonium salt; extracts or enriched fractions of such extracts such as extracts of Thaumatococcus or Stevia ssp., particularly Stevia rebaudiana, stevia leaf extract, swingle extracts, particularly Momordica or Siratia grosvenorii or Luo-Han-Guo, extracts of Glycerrhyzia ssp., particularly Glycerrhyzia glabra or Glycerrhyzia uralensis, extracts of Rubus ssp., particularly Rubus suavissimus or Rubus chingii comprising rubusoside, extracts of Mycetia balansae, preferably comprising balansin A and/or balansin B and,
synthetic sweet tasting substances, preferably selected from the group consisting of magap, sodium cyclamate or other physiologically acceptable salts of cyclamic acid, acesulfam K, saccharin, saccharin sodium salt, aspartam, superaspartam, neotam, alitam, advantam, perillartin, sucralose, lugduname, carrelame, sucrononate and sucrooctate,
or mixtures thereof,
and optionally
(iii) combining the compound or mixture of compounds of formula (I) or the one or more sweet tasting substance(s) or the mixture obtained in step (ii) with one or more sweet taste modulating substances selected from the group consisting of hesperetin, hesperetin dihydrochalcone, naringenin, phloretin, eriodictyol, homoeriodictyol, phyllodulcin, neohesperidindihydrochalkon, naringindihydrochalkon, phloretin, extracts of Hydrangea macrophylla ssp. serrata, in particular var. Oamacha, Amacha or Amagi amacha comprising active amounts of phyllodulcin.

Step (iii) can be performed before step (ii), i.e. by combining the one or more sweet taste modulating substances with the compound(s) of formula (I) from step (i) and then combining the resulting mixture with the one or more sweet tasting substance(s) in step (ii), or the one or more sweet tasting substance(s) can be combined with the one or more sweet taste modulating substances before they are combined with the compound(s) of formula (I) in step (ii).

Particularly preferably, in the method according to any embodiment described above, the one or more sweet tasting substance(s) is/are selected from the group consisting of sucrose, fructose, glucose, steviosides, rebaudiosides, rebaudioside A, rebaudioside M, mono-, di-, tri- or tetra-alpha-glycosylated steviosides or rebaudiosides, rubusoside, mono, di-, tri- or tetra-alpha-glycosylated rubusosides.

Further preferably, in the method according to any embodiment described above, in step (ii), compound (1) is combined with one or more sweet tasting substance(s) selected from the group consisting of sucrose, fructose, glucose, steviosides, rebaudiosides, rebaudioside A, rebaudioside M, mono-, di-, tri- or tetra-alpha-glycosylated steviosides or rebaudiosides, rubusoside, mono-, di-, tri- or tetra-alpha-glycosylated rubusosides.

Further preferable is a method as described above, wherein in step (ii), compound (1) and one or more sweet tasting substances selected from the group consisting of sucrose, fructose, glucose, stevisodes, rebaudiosides, rebaudioside A, rebaudioside M, mono-, di-, tri- or tetra-alpha-glycosylated steviosides or rebaudiosides, rubusoside, mono-, di-, tri- or tetra-alpha-glycosylated rubusosides are combined and in step (iii), compound (1) and the one or more sweet tasting substance(s) are further combined with one or more sweet taste modulating substance(s) selected from the group consisting of phyllodulcin, hersperetin, hesperetindihydrochalkon and phloretin or extracts of Hydrangea macrophylla ssp. serrata, in particular var. Oamacha, Amacha or Amagi amacha comprising active amounts of phyllodulcin.

In one embodiment of the method described above, the compound or mixture of two or more compounds of formula (I) and/or the one or more sweet tasting substance(s) and optionally the one or more sweet taste modulating substance(a) are combined with further components to form a product as described above.

It was found out that the compounds of formula (I) can be produced by chemical or enzymatic alkanoylation of trilobatin or a plant extract comprising trilobatin, in particular an extract from Lithocarpus species such as Averrhoa carambola, L. litseifolius, L. polystachyus and L. pachyphyllus or from Oxytropis myriophylla, Homalium stenophyllum, Malus trilobata, Symplocos species such as S. lancifolia, S. spicata and S. vacciniifolia, Vitis piasezkii and Vitis saccharifera. The best results can be obtained by extracting L. litseifolius, L. polystachyus and Malus trilobata. In Lithocarpus litseifolius and Lithocarpus polystachyus particularly large amounts of trilobatin are found in the leaves, while for Malus trilobata, extraction of root material gives the best results.

The present invention also relates to a method of producing a compound or a mixture of two or more compounds of formula (I) comprising the steps:
(i) providing trilobatin or an extract from Lithocarpus litseifolius or Lithocarpus polystachyus leaf material or an extract from Malus trilobata root material; and
(ii) subjecting the trilobatin or the extract from step (i) to a chemical or enzymatic alkanoylation.

To provide an extract from the plant materials in step (i), plant material, in particular leafs of Lithocarpus litseifolius or Lithocarpus polystachyus is subjected to extraction with a solvent at a temperature from 0° C. to the boiling point of the respective solvent. For the extraction, the ratio of plant material:solvent may be between 1:10 to 1:30, preferably 1:15 to 1:25. Preferably, the solvent is selected from the group consisting of water, subcritical or supercritical water, methanol, ethanol and mixtures thereof. The solvent may be removed and the extract purified by solid-phase adsorption. The adsorbent may be e.g. polystyrene or a mixture of polystyrene and other components. The purification with polystyrene resin yields extracts with around 25% trilobatin. Preferably, water and ethanol are used as solvent with a weight ratio of water:ethanol of at least 1:3, the extraction temperature being between 30 and 50° C., preferably 35 to 45° C. Alternatively, extracts from Lithocarpus litseifolius, Lithocarpus polystachyus and Malus trilobata plant material are commercially available.

The tribobatin or extract from step (i) of the method described above is then acylated either chemically or enzymatically. In a preferred embodiment of the method described above, in step (ii), the trilobatin or the extract from step (i) is acetylated with acetic acid anhydride as shown in Scheme 1.

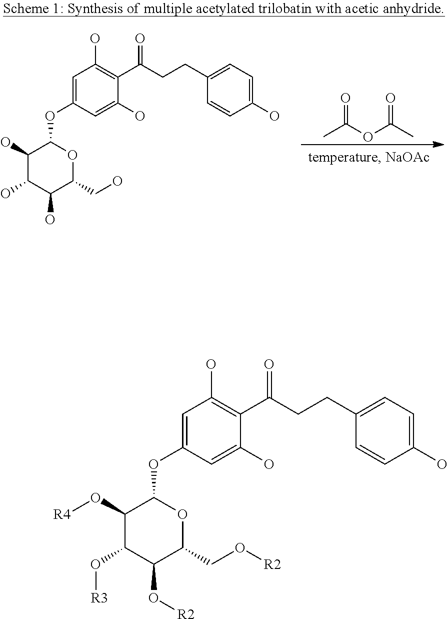

Alternatively, acetylated trilobatin can be obtained by enzymatic acylation. Therefore, in another preferred embodiment of the method described above, in step (ii), the trilobatin or the extract from step (i) is incubated with an acyl donor and a lipase.

Acyl donors may be selected form the group consisting of triacetin, tripropionin, tributyrin, acetic acid, acetic anhydride, ethyl acetate, ethyl propionate and ethyl butanoate.

The lipase used in step (ii) of the method described above may be obtained from a microorganism selected from the group consisting of Candida antarctica A, Candida antarctica B, Candida rugosa, Burkholderia cepacia, Rhizopus sp., Rhizomucor miehei, Mucor javanicus, Yarrowia lypolytica, Geotrichum candidum, Aspergillus niger, Aspergillus oryzae, Pseudomonas alcaligenes, Pseudomonas mendocina, Thermomyces lanuginosus and Chromobacterium viscosum. These lipases are commercially available and are provided with instructions for the use by the manufacturer.

A mixture of compounds may be obtained in the method described above, when the acylation is performed enzymatically. In particular, a mixture may be obtained in step (ii), which comprises or consists of phloridizin, trilobatin, 6″-O-acetylphloridizin, 6″-O-acetyltrilobatin (compound (1)), 3″,6″-O-diacetylphloridin and 3″,6″-O-diacetyltrilobatin (compound (5)). In case an extract from Lithocarpus litseifolius or Lithocarpus polystachyus leaf material or from Malus trilobata root material is used, the mixture is significantly different from the mixture obtained from pure trilobatin.

The present invention also relates to a compound or mixture of two or more compounds of formula (I) obtained or obtainable by a method according to any of the embodiments described above.

Furthermore, the present invention also relates to the use of compound ( ) as a flavor, in particular as a sweet tasting and/or sweet taste modulating substance.

The taste and the taste modulating properties of compound (1) have not been described in the prior art.

Finally, the present invention also relates to a compound or mixture of compounds of formula (I) wherein R1, R2, R3 and R4 are defined as shown in the following table:

| Compound | R1 | R2 | R3 | R4 |
|---|---|---|---|---|
| (2) | H | H | acetyl | H |
| (3) | propionyl | H | H | H |
| (4) | H | H | propionyl | H |
| (5) | acetyl | H | acetyl | H |
| (6) | H | acetyl | H | H |
| (8) | H | propionyl | H | H |
| (9) | H | H | H | propionyl |
| (10) | butanoyl | H | H | H |
| (11) | H | butanoyl | H | H |
| (12) | H | H | butanoyl | H |
| (13) | H | H | H | butanoyl |
| (14) | acetyl | acetyl | H | H |
| (15) | acetyl | H | H | acetyl |
| (16) | H | acetyl | acetyl | H |
| (17) | H | acetyl | H | acetyl |
| (18) | H | H | acetyl | acetyl |
| (19) | acetyl | acetyl | acetyl | H |
| (20) | H | acetyl | acetyl | acetyl |
| (21) | acetyl | H | acetyl | acetyl |
| (22) | acetyl | acetyl | H | acetyl |
| (24) | propionyl | propionyl | H | H |
| (25) | propionyl | H | propionyl | H |
| (26) | propionyl | H | H | propionyl |
| (27) | H | propionyl | propionyl | H |
| (28) | H | propionyl | H | propionyl |
| (29) | H | H | propionyl | propionyl |
| (30) | H | propionyl | propionyl | propionyl |
| (31) | propionyl | H | propionyl | propionyl |
| (32) | propionyl | propionyl | H | propionyl |
| (33) | propionyl | propionyl | propionyl | H |
| (34) | propionyl | propionyl | propionyl | propionyl |
| (35) | butanoyl | butanoyl | H | H |
| (36) | butanoyl | H | butanoyl | H |
| (37) | butanoyl | H | H | butanoyl |
| (38) | H | butanoyl | butanoyl | H |
| (39) | H | butanoyl | H | butanoyl |
| (40) | H | H | butanoyl | butanoyl |
| (41) | butanoyl | butanoyl | butanoyl | H |
| (42) | butanoyl | butanoyl | H | butanoyl |
| (43) | butanoyl | H | butanoyl | butanoyl |
| (44) | H | butanoyl | butanoyl | butanoyl |
| (45) | butanoyl | butanoyl | butanoyl | butanoyl |

These compounds have not been described in the prior art and no information was therefore available on their taste and sweet modulation properties.

The invention is further characterized by illustrative, non-limiting examples.

### SHORT DESCRIPTION OF THE FIGURES

FIG. 1 shows the Liquid Chromatography-Mass Spectrometry ((LC-MS) chromatogram of acetylated products of trilobatin and phloridizin of Lithocarpus extract.

FIG. 2 shows the Liquid Chromatography-High Resolution Mass Spectrometry (LC-HRMS) analysis of a mixture of isolated diacetylated trilobatins. Charged aerosol detector signal is shown. Compound #1 represents 6″-O-acetyltrilobatin und compounds #2-6 represent different dicacetyltrilobatins.

### EXAMPLE 1: SENSORY IMPACT OF TRILOBATIN AND COMPOUND (1)

The sensory impact of trilobatin and compound (1) have been compared by a trained sensory panel (Table 1) using a series of paired comparison tests. The samples were coded and randomized (referred to as Duo test):

TABLE 1
Duo test of compounds against 5% sucrose solution

| | number | | sweet modul. | |
|---|---|---|---|---|
| | of | dosage | activity | |
| compound | panelists | (mg/kg) | (%) | t-test |
| trilobatin | 20 | 50 | 1.66 | 0.857 |
| compound (1) | 20 | 50 | 27.54 | 0.006 |

Each of compound (1) and trilobatin has been compared with a 5% sucrose solution in water. Surprisingly, 6″-O acetylated trilobatin showed significant (p=0.006) sweet modulating activity whereas trilobatin did not show a significant sensory effect By addition of 50 mg/kg compound (1) the taste of the 5% sucrose solution was perceived as 27.5% sweeter than without addition.

### EXAMPLE 2: ENZYMATIC ACETYLATION OF TRILOBATIN AND L. LITSEIFOLIUS EXTRACT

A Lithocarpus litseifolius leaf extract containing 7% phloridizin and 47% trilobatin as well as 85% pure trilobatin were enzymatically (80° C., 4.5 h, Novozyme 435) treated with lipase from Candida Antarctica in triacetin. Two mixtures with the following concentration of compounds were obtained:

TABLE 2
Composition of enzymatically
treated L. litseifolius extract and trilobatin

| | L. litseifolius | | trilobatin | |
|---|---|---|---|---|
| | | 0.25% | | 0.25% |
| | amount | in water | amount | in water |
| compound | [%] | [mg/kg] | [%] | [mg/kg] |
| phloridizin | 0.52 | 12.88 | 0.05 | 1.20 |
| trilobatin | 2.29 | 57.30 | 0.12 | 2.92 |
| 6″-O-acetylphloridizin | 0.37 | 9.23 | 0.16 | 4.03 |
| 6″-O-acetyltrilobatin | | | | |
| (compound (1)) | 1.63 | 40.63 | 0.76 | 19.10 |
| 3″,6″-O-diacetylphloridizin | 0.22 | 5.56 | 0.36 | 9.04 |
| 3″,6″-O-diacetyltrilobatin | | | | |
| (compound (5)) | 0.38 | 9.58 | 0.44 | 10.91 |

Table 2 shows that a specific composition of phloretin derivatives can be obtained by enzymatic transfer of acetyl group from triacetin to phloridizin and trilobatin respectively: 85% pure trilobatin was converted with high yield >90% to mono-, di and triacetylated trilobatin with almost equal amount of monoacetylated and diacetylated aglycons. In comparison, trilobatin from L. litseifolius extract in triacetin was only converted by less than 60% yielding 6″-O-acetyltrilobatin as main component and <10% diacetylated phloridizin and trilobatin. Despite the differences in the degree of acetylation, comparative sensory evaluation of both samples revealed equal sweet modulating activity.

### EXAMPLE 3: CHEMICAL SYNTHESIS OF MULTIPLE ACETYLATED TRILOBATIN

2.5 g/L. litseifolius extract (18% trilobatin) was dissolved in 20 mL acetic anhydride. 4 g of sodium acetate was added and the mixture was slowly refluxed for 2 hours. The reaction mixture was cooled down, 50 mL water were added to hydrolyse excess of acetic anhydride and the crude product was finally liquid-liquid extracted by adding another 100 mL water and ethyl acetate (three times with phase ratio 1:1). The organic phase was neutralized with sodium carbonate solution, dried and concentrated. Trilobatin was completely converted to multiple acetylated trilobatin and the mixture showed sweet taste in water at 200 mg/kg dosage.

### EXAMPLE 4: ENZYMATIC ACETYLATION

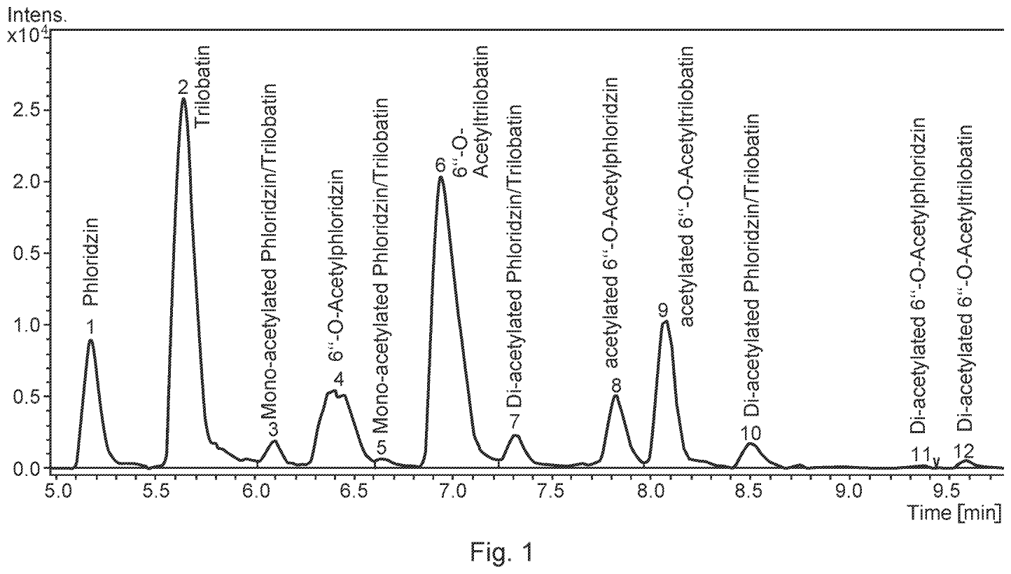

Acetylated Trilobatin can be obtained via enzymatic acetylation using an acetyl donor and a lipase. Trilobatin and Lithocarpus extract (50% Trilobatin) were suspended in triacetin at concentrations of 100 g/L and 200 g/L, respectively, and dissolved via incubation at 70° C. Lipase Novozym 435 (Novozymes, Lyngby, Denmark) was added to the solutions at concentrations of 50 g/L and incubated under agitation for 4.5 h at 70° C. After incubation lipase was removed via filtration. Samples were analysed via HPLC-MS and acetylated products of acetylated Lithocarpus extract are shown in FIG. 1.

### EXAMPLE 5: ENZYMATIC ACETYLATION OF L. LITSEIFOLIUS LEAF EXTRACT

200 g/l L. litseifolius leaf extract (47% trilobatin, 7% phloridizin) was dissolved in triacetin and reacted with lipase Novozyme 435 for 6 h at 80° C. After cooling the mixture, the solution was filtrated. The solution contained 4.2% compound 1. The intrinsic sweetness of the solution was determined by a Duo test comparing different concentrations of this solution with a 1.5% sucrose sample. It turned out that 0.15% of the solution (containing 63 mg/kg compound 1) was as sweet as 1.5% sucrose. As a consequence, a solution containing 63 mg/kg compound 1 is much sweeter (like 1.5% sucrose) than 100 mg/kg trilobatin (only “weakly sweet” comparable to 0.5% sucrose solution) as disclosed in WO2008148239.

### EXAMPLE 6: ENZYMATIC ACETYLATION OF TRILOBATIN

150 g/l Trilobatin (78% purity) was dissolved in triacetin and reacted with lipase Novozyme 435 for 6 h at 80° C. After cooling the mixture, the solution was filtrated. The solution contained 4.5% compound 1 and 3.9% diacetylated phloretin-4′-glucoside (such as compounds 5). A Duo test based on a 2% sucrose solution was performed to determine the threshold concentration above sweet modulating property could be detected. A concentration of 0.025% in 2% sucrose solution was significantly sweeter than the 2% sucrose solution without addition. As a consequence, the sum of 23 mg/kg of mono and diacetylated phloretin-4′-glucoside induced a perceivable sweet modulating activity.

### EXAMPLE 7: CHARACTERIZATION OF DIACETYLATED PHLORETIN-4′-GLUCOSIDES

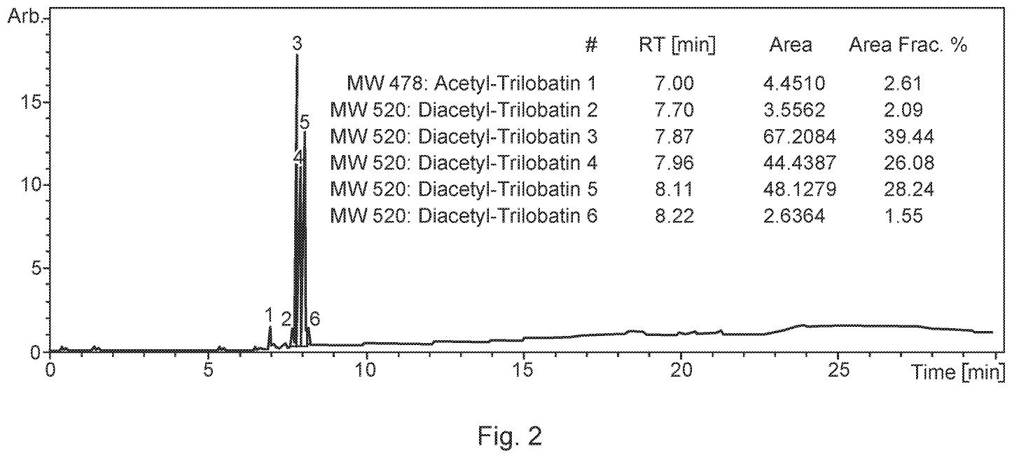

Diacetylated phloretin-4′-glucosides (such as compound 5) were additionally isolated from a reaction mixture as disclosed in example 2 and characterized by liquid chromatography (Waters Acquity UPLC system, Waters) coupled with high-resolution mass spectrometry (microTOFQII, Bruker) and charged aerosol detector (Corona Veo, Thermo). Chromatographic separation was carried out on a C-18 column (Kinetex, 100 mm×2.1 mm, 1.7 μm; Phenomenex) at a temperature of 50° C. and a flow rate of 0.55 mL/min using an acetonitrile/water gradient (FIG. 2). The analysis of the mixture shows that three main stereoisomers of diacetylated trilobatin with molecular mass 520 are formed by enzymatic transesterification (no. 3, 4, 5). Surprisingly it was found by tasting of 100 mg/kg of this isolate in water with five trained flavorists a sweet and bitter taste. Therefore, acetylation must take place at certain positions to reveal desired sensory performance

### EXAMPLE 8: INTRINSIC SWEETNESS OF COMPOUND (1)

The intrinsic sweetness of pure compound (1) (>95%) was determined by a panel of 20 trained panellists. Thereby, a 1.5% sucrose solution was compared by a Duo test with 65 mg/kg of compound (1) in water. Statistical elaboration revealed no statistically significant difference (p<0.05) between the samples and therefore 65 mg/kg of compound 1 tastes as sweet as a 1.5% sucrose solution. As a consequence, a solution containing 65 mg/kg compound 1 is much sweeter (like 1.5% sucrose) than 100 mg/kg trilobatin (only “weakly sweet” comparable to 0.5% sucrose solution) as disclosed in WO2008148239. The intrinsic sweetness of compound (1) in a mixture of trilobatin and diacetyl trilobatins was similar than of pure compound (1).

### EXAMPLE 9: SENSORY PROFILE OF SUGAR SOLUTION WITH ADDED COMPOUNDS

The compound (1) improves the overall taste of sweetened solutions as shown in table 3.

TABLE 3
Sensory profile of sugar solution with added compounds

| | Impact | Intensity | Mouthfeel | Aftertaste |
|---|---|---|---|---|
| 5% sucrose solution (A1) | 3 | 4 | 4 | 1 |
| A1 + trilobatin 80% | | | | |
| dosage.: 80 mg/kg | 3 | 4 | 5 | 1 |
| A1 + compound (1) 81% | 4 | 6 | 5 | 1 |
| dosage: 80 mg/kg | | | | |

### EXAMPLE 10

A dose of 10 mg/kg G92828 (equals 16 mg/kg of compound (1)) was combined with RS (20% Rubusosid, a sweet tasting substance as described above), PD (2.5% Phyllodulcin), HT (>85% Hesperetin) and PH (>98% Phloretin, all three sweet taste modulating substances as described above) at doses of 200, 120, 10 and 30 mg/kg respectively and the mixture was applied to restore the overall and sweet taste of an uncarbonated orange softdrink matrix with reduced sugar content (regular: 10-11%). Different descriptors relevant for the overall liking of an orange softdrink were applied and the differences in intensities of each descriptor (Δ) of FMP (A) to FMP+ compound (1) (B) were determined by a panel of five flavorists (n=5) and given as intensities from 0-9 as shown in Tables 4-12.

The panelists were asked to neutralise with tap water between each sample and the intensity of the descriptors of each sample were defined before tasting the next sample. The tasting of each RS, PD, HT or PH (A) were followed by the samples with added RS, PD, HT or PH+compound (1) (B) with each being compared to the intensities of the base.

TABLE 4
Difference (A) of onset sweetness between different
sweet tasting substance (RS) or sweet taste
modulating substances (PD, HT, PH) (A) and sweet
tasting substance (RS) or sweet modulating substances
(PD, HT, PH) + 20 mg/kg G92828 (B) in orange
softdrink base (5% sucrose + 0.15% citric acid + 0.2%
orange oil washed (product number 332980, Symrise))

| | Sweet tasting | | | | |
|---|---|---|---|---|---|
| | substance (RS) | | | | |
| | or sweet taste | | | | |
| | modulating | | | | |
| | substances | Dose | Onset sweetness (0-9) | | |
| Nr | (PD, HT, PH) | [mg/kg] | A | B | Δ |
| 0 | Base | | 4.0 | 4.5 | 0.5 |
| 1 | RS | 200 | 5.0 | 5.5 | 0.5 |
| 2 | PD | 120 | 4.5 | 5.5 | 1.0 |
| 3 | HT | 10 | 5.0 | 6.0 | 1.0 |
| 4 | PH | 30 | 5.0 | 6.0 | 1.0 |

TABLE 5
Difference (Δ) of overall sweetness between different sweet
tasting substance (RS) or sweet taste modulating substances
(PD, HT, PH) (A) and sweet tasting substance (RS) or sweet
taste modulating substances (PD, HT, PH) + 20 mg/kg
G92828 (B) in orange softdrink base (5% sucrose + 0.15%
citric acid + 0.2% orange oil washed (product number
332980, Symrise))

| | Sweet tasting | | | | |
|---|---|---|---|---|---|
| | substance (RS) or | | Overall | | |
| | sweet taste modulating | | sweetness (0-9) | | |
| Nr | substances (PD, HT, PH) | Dose [mg/kg] | A | B | Δ |
| 0 | Base | | 5.0 | 6.0 | 1.0 |
| 1 | RS | 200 | 6.0 | 7.0 | 1.0 |
| 2 | PD | 120 | 6.0 | 7.5 | 1.5 |
| 3 | HT | 10 | 6.0 | 7.5 | 1.5 |
| 4 | PH | 30 | 6.5 | 6.5 | 0.0 |

TABLE 6
Difference (Δ) of orange flavor between different sweet tasting substance
(RS) or sweet taste modulating substances (PD, HT, PH) (A) and sweet
tasting substance (RS) or sweet taste modulating substances (PD, HT,
PH) + 20 mg/kg G92828 (B) in orange soft-drink base (5% sucrose +
0.15% citric acid + 0.2% orange oil washed (product number 332980,
Symrise))

| | Sweet tasting substance | | | | |
|---|---|---|---|---|---|
| | (RS) or sweet taste modulating | | orange (0-9) | | |
| Nr | substances (PD, HT, PH) | Dose [mg/kg] | A | B | Δ |
| 0 | Base | | 5.0 | 5.0 | 5.0 |
| 1 | RS | 200 | 4.0 | 4.0 | 0.0 |
| 2 | PD | 120 | 4.0 | 4.0 | 0.0 |
| 3 | HT | 10 | 5.0 | 5.0 | 0.0 |
| 4 | PH | 30 | 5.0 | 5.0 | 0.0 |

TABLE 7
Difference (Δ) of fruity character between different sweet tasting
substance (RS) or sweet taste modulating substances (PD, HT,
PH) (A) and sweet tasting substance (RS) or sweet taste
modulating substances (PD, HT, PH) + 20 mg/kg G92828 (B)
in orange soft-drink base (5% sucrose + 0.15% citric acid + 0.2%
332980 orange oil washed (product number 332980, Symrise))

| | Sweet tasting substance | | | | |
|---|---|---|---|---|---|
| | (RS) or sweet taste modulating | | fruity (0-9) | | |
| Nr | substances (PD, HT, PH) | Dose [mg/kg] | A | B | Δ |
| 0 | Base | | 5.0 | 5.0 | 0.0 |
| 1 | RS | 200 | 6.0 | 6.5 | 0.5 |
| 2 | PD | 120 | 5.0 | 5.5 | 0.5 |
| 3 | HT | 10 | 5.0 | 5.0 | 0.0 |
| 4 | PH | 30 | 5.5 | 5.5 | 0.0 |

TABLE 8
Difference (Δ) of juicy character between different sweet tasting
substance (RS) or sweet taste modulating substances (PD, HT,
PH) (A) and sweet tasting substance (RS) or sweet taste
modulating substances (PD, HT, PH) + 20 mg/kg G92828 (B)
in orange soft-drink base (5% sucrose + 0.15% citric acid + 0.2%
orange oil washed (product number 332980, Symrise))

| | Sweet tasting substance | | | | |
|---|---|---|---|---|---|
| | (RS) or sweet taste modulating | | juicy (0-9) | | |
| Nr | substances (PD, HT, PH) | Dose [mg/kg] | A | B | Δ |
| 0 | Base | | 4.0 | 4.0 | 0.0 |
| 1 | RS | 200 | 5.0 | 5.5 | 0.5 |
| 2 | PD | 120 | 3.5 | 4.5 | 1.0 |
| 3 | HT | 10 | 4.0 | 5.0 | 1.0 |
| 4 | PH | 30 | 5.0 | 5.5 | 0.5 |

TABLE 9
Difference (Δ) of acidity between sweet tasting substance (RS) or sweet
taste modulating substances (PD, HT, PH) (A) and sweet tasting
substance (RS) or sweet taste modulating substances (PD, HT, PH) + 20
mg/kg G92828 (B) in orange softdrink base (5% sucrose + 0.15% citric
acid + 0.2% orange oil washed (product number 332980, Symrise))

| | Sweet tasting substance | | | | |
|---|---|---|---|---|---|
| | (RS) or sweet taste modulating | | acidity (0-9) | | |
| Nr | substances (PD, HT, PH) | Dose [mg/kg] | A | B | Δ |
| 0 | Base | | 4.0 | 3.5 | −0.5 |
| 1 | RS | 200 | 3.0 | 2.5 | −0.5 |
| 2 | PD | 120 | 3.5 | 3.5 | 0.0 |
| 3 | HT | 10 | 4.0 | 3.5 | −0.5 |
| 4 | PH | 30 | 3.5 | 3.0 | −0.5 |

TABLE 10
Difference (Δ) of mouthfeel between different sweet tasting substance
(RS) or sweet taste modulating substances (PD, HT, PH) (A) and sweet
tasting substance (RS) or sweet taste modulating substances (PD, HT,
PH) + 20 mg/kg G92828 (B) in orange soft-drink base (5% sucrose +
0.15% citric acid + 0.2% orange oil washed (product number 332980,
Symrise))

| | Sweet tasting substance | | mouthfeel | | |
|---|---|---|---|---|---|
| | (RS) or sweet taste modulating | Dose | (0-9) | | |
| Nr | substances (PD, HT, PH) | [mg/kg] | A | B | Δ |
| 0 | Base | | 3.5 | 4.0 | 0.5 |
| 1 | RS | 200 | 5.0 | 5.5 | 0.5 |
| 2 | PD | 120 | 4.0 | 5.0 | 1.0 |
| 3 | HT | 10 | 4.0 | 4.5 | 0.5 |
| 4 | PH | 30 | 4.0 | 5.0 | 1.0 |

TABLE 11
Difference (Δ) of lingering sweetness between different sweet tasting
substance (RS) or sweet taste modulating substances (PD, HT, PH) (A)
and sweet tasting substance (RS) or sweet taste modulating substances
(PD, HT, PH) + 20 mg/kg G92828 (B) in orange softdrink base (5%
sucrose + 0.15% citric acid + 0.2% orange oil washed (product
number 332980, Symrise))

| | Sweet tasting substance | | | | |
|---|---|---|---|---|---|
| | (RS) or sweet taste | | Lingering | | |
| | modulating substances | | sweetness (0-9) | | |
| Nr | (PD, HT, PH) | Dose [mg/kg] | A | B | Δ |
| 0 | Base | | 0.0 | 0.0 | 0.0 |
| 1 | RS | 200 | 1.0 | 1.5 | 0.5 |
| 2 | PD | 120 | 2.0 | 1.5 | −0.5 |
| 3 | HT | 10 | 0.0 | 0.0 | 0.0 |
| 4 | PH | 30 | 0.0 | 0.0 | 0.0 |

TABLE 12
Difference (Δ) of off notes between different sweet tasting substance
(RS) or sweet taste modulating substances (PD, HT, PH) (A) and
sweet tasting substance (RS) or sweet taste modulating substances
(PD, HT, PH) + 20 mg/kg G92828 (B) in orange soft-drink base (5%
sucrose + 0.15% citric acid + 0.2% orange oil washed (product
number 332980, Symrise))

| | F Sweet tasting substance | | | | |
|---|---|---|---|---|---|
| | (RS) or sweet taste modulating | Dose | Off note (0-9) | | |
| Nr | substances (PD, HT, PH) | [mg/kg] | A | B | Δ |
| 0 | Base | | 0.0 | 0.0 | 0.0 |
| 1 | RS | 200 | 2.0 | 2.0 | 0.0 |
| 2 | PD | 120 | 2.0 | 2.0 | 0.0 |
| 3 | HT | 10 | 0.0 | 0.0 | 0.0 |
| 4 | PH | 30 | 0.0 | 0.0 | 0.0 |

Table 4 shows that 6″-O-acetyltrilobatin (compound (1)) increases the onset sweetness in combination with RS, PD, HT and PH, with best effects for PD, HT and PH.

The overall sweetness (Table 5) is increased by combination of 6″-O-acetyltrilobatin with RS, PD and HT, while proving especially effective for combination with PD and HT. A combination with PH is showing no increase in overall sweetness in comparison to PH but an improvement in onset sweetness. The taste of the lingering aftertaste of PD is slightly reduced, while a combination with RS shows a slight increase in lingering aftertaste.

The use of acetyltrilobatin shows no impact on the intensity of orange flavor (Table 6) or off notes (Table 12) for the soft drink base, as well as in combination with RS, PD, HT and PH, while increasing the juicy character in combination with each tested FMP (Table 8) and also increasing fruity notes in combination with RS and PD (Table 7).

The use of 6″-O-acetyltrilobatin further improves the mouthfeel of the test base and every tested FMP (Table 10) while reducing perceived acidity of the base, RS, HT and PH but not with PD (Table 9).

These results clearly show positive effects for a combination with each tested sweet tasting substance or sweet modulating substance. These effects do not appear to be depending on each other as well, as some of the results show:

The increase in overall sweetness is strongest for combinations with PD and HT, while a combination with PH shows no difference in overall sweetness but similar increase in onset sweetness as a combination of 6″-O-acetyltrilobatin with PD and HT. The increase in overall sweetness does not impact the lingering aftertaste in combination with HT and the intensity of lingering aftertaste of PD is even decreased, while these two FMPs show most increase in overall sweetness in combination with 6″-O-acetyltrilobatin.

The improvement of mouthfeel is best with PD and PH, while a combination with PH is showing no increase in overall sweetness and a combination with PD shows no reduction in acidity. A slighter improvement of mouthfeel and acidity was further determined for the use of 6″-O-acetyltrilobatin in test base alone.

While PD alone is reducing the intensity of juicy notes of the test base, a combination of PD with 6″-O-acetyltrilobatin increases them above test base levels. While having no effect on the test base alone, the use of 6″-O-acetyltrilobatin shows even more overall intensity of juicy notes in combinations with RS, HT and PH and most increase of intensity with HT and PD.

### APPLICATION EXAMPLES

### Application Example 1

### Spray-Dried Preparation as a Semi-Finished Product for Flavouring of Finished Products

| Ingredient | Use in % by weight | | | | | |
|---|---|---|---|---|---|---|
| Preparation | A | B | C | D | E | F |
| Drinking water | 60.8 | 60.8 | 60.8 | 60.8 | 60.8 | 60.8 |
| Maltodextrin from wheat | 31.5 | 29.7 | 28.8 | 27.0 | 30.2 | 30.0 |
| Gum Arabic | 6.1 | 6.1 | 6.1 | 6.1 | 6.1 | 6.1 |
| 6″-O-Acetyltrilobatin, compound (1) | 1.6 | 1.2 | 1.0 | 0.6 | 1.3 | 1.3 |
| Hesperetin | — | 2.2 | — | — | 1.1 | 1.1 |
| Homoeriodictyol-sodium salt | — | — | — | 5.5 | — | — |
| Phloretin | — | — | 3.3 | — | — | — |
| Hesperetindihydrochalkon | — | — | — | — | 0.5 | — |
| Phyllodulcin (dry Oamacha extract containing | — | — | — | — | — | 0.7 |
| 70% Phyllodulcin) | | | | | | |

The drinking water is placed in a container and maltodextrin and gum arabic is dissolved in it. Then the flavouring is emulsified in the carrier solution with a Turrax. The temperature of the spray solution should not exceed 30° C. The mixture is then spray-dried (inlet nominal temperature: 185-195° C., outlet nominal temperature: 70-75° C.).

### APPLICATION EXAMPLE 2

Combination with Sweeteners
90 g sucrose and 10 g tagatose are added to 0.5 g of a spray-dried semi-finished product from application example 1 (according to preparation A) and mixed. The product can for example be used as a sweetener with a bitter masking effect for coffee or tea.

### APPLICATION EXAMPLE 3

### Chewing Gum

| Part | Ingredient | % b.w. |
|---|---|---|
| A | Chewing gum base from “Jagum T” company | 30.9 |
| B | Sorbitol, powdered | 39.0 |
| | Isomalt ® (Palatinit GmbH) | 9.5 |
| | Xylitol | 2.0 |
| | Mannitol | 3.0 |
| | Rebaudioside A 98% | 0.2 |
| | Emulgum ® (Colloides Naturels, Inc.) | 0.3 |
| C | Sorbitol, 70% | 14.0 |
| | Glycerin | 1.0 |
| D | Flavouring, containing 3.2% 6″-O-acetyltrilobatin, | 0.1 |
| | compound (1) based on the total weight of the | |
| | flavouring (preparation A from Application | |
| | Example 1) | |

Parts A to D are mixed and kneaded intensively. The raw mass can be processed by way of example in the form of thin strips into ready-to-consume chewing gum.

### APPLICATION EXAMPLE 4

### Toothpaste

| Part | Ingredient | % b.w. |
|---|---|---|
| A | Demineralized water | 22.00 |
| | Sorbitol (70%) | 46.00 |
| | Solbrol ® M, sodium salt (Bayer AG, p- | 0.15 |
| | hydroxybenzoic acid alkyl ester) | |
| | Trisodium phosphate | 0.10 |
| | Rebaudioside A, 98% | 0.10 |
| | Sodium monofluorophosphate | 1.12 |
| | Polyethylene glycol 1500 | 5.00 |
| B | Sident 9 (abrasive silicon dioxide) | 10.00 |
| | Sident 22 S (thickening silicon dioxide) | 8.00 |
| | Sodium carboxymethylcellulose | 0.90 |
| | Titanium dioxide | 0.50 |
| C | Demineralized water | 4.63 |
| | Sodium lauryl sulfate | 1.50 |
| D | Flavouring, containing 0.1% 6″-O-acetyltrilobatin, | 1.00 |
| | compound (1) based on the total weight of the | |
| | flavouring | |

The ingredients of parts A and B are in each case pre-mixed separately and stirred well under a vacuum at 25-30° C. for 30 minutes. Part C is pre-mixed and added to A and B; D is added and the mixture stirred well under a vacuum at 25-30° C. for 30 minutes. After pressure relief the toothpaste is finished and can be filled.

### APPLICATION EXAMPLE 5

### Sugar-Free Hard Boiled Candy

| | Content (%) | | | |
|---|---|---|---|---|
| Ingredient | A | B | C | D |
| Palatinite, Type M | 75.00 | 74.00 | 75.50 | 75.00 |
| Citric acid | — | 1.00 | 0.50 | — |
| Colouring, yellow | — | 0.01 | — | — |
| Colouring, red | — | — | 0.01 | — |
| Colouring, blue | 0.01 | — | — | 0.01 |
| Peppermint flavouring | 0.1 | — | — | 0.1 |
| Lemon flavouring | — | 0.1 | — | — |
| Red fruit flavouring | — | — | 0.1 | — |
| 6″-O-acetyltrilobatin, | 0.002 | 0.0010 | 0.003 | 0.002 |
| compound (1) | | | | |
| Rebaudioside A 98% | — | 0.040 | — | 0.040 |
| hesperetin dihydrochalcone | — | — | 0.001 | — |
| Hesperetin | — | 0.001 | — | 0.001 |
| Phloretin | — | 0.002 | — | — |

| | |
|---|---|
| Water | ad 100 |

Palatinite was mixed with water and the mixture melted at 165° C. and then cooled to 115° C. The other ingredients were added and after mixing cast into moulds, following hardening removed from the moulds and then individually packaged.

### APPLICATION EXAMPLE 6

### Sugar-Reduced Tomato Ketchup

A: Comparative preparation with sugar
B: Comparative preparation with reduced sugar content (compared to A)
C-H: Preparations according to the invention with reduced sugar content (compared to A) and 6″-O-acetyltrilobatin, compound (1)

| | Preparation (amounts in % by weight) | | | | | | | |
|---|---|---|---|---|---|---|---|---|
| Ingredient | A | B | C | D | E | F | G | H |
| Common salt | 2.0 | 2.0 | 2.0 | 2.0 | 2.0 | 2.0 | 2.0 | 2.0 |
| Starch, Farinex WM 55 | 1.0 | 1.0 | 1.0 | 1.0 | 1.0. | 1.0 | 1.0 | 1.0 |
| Sucrose | 12.0 | 9.6 | 9.2 | 8.4 | 9.6 | 9.6 | 8.4 | 8.4 |
| Tomato concentrate x 2 | 40.0 | 40.0 | 40.0 | 40.0 | 30.0 | 30.0 | 30.0 | 30.0 |
| Glucose syrup 80 Brix | 18.0 | 18.0 | 18.0 | 18.0 | 18.0 | 18.0 | 18.0 | 18.0 |
| Spirit vinegar 10% | 7.0 | 7.0 | 7.0 | 7.0 | 3.0 | 3.0 | 3.0 | 3.0 |
| 6″-O-Acetyltrilobatin, compound | | | 0.2 | 0.15 | 0.15 | 0.25 | 0.1 | 0.1 |
| (1), 0.5% in 1,2-propylene glycol | | | | | | | | |
| Hesperetin dihydrochalcone (I) | | | | 0.1 | | 0.05 | 0.05 | |
| 0.5% in 1,2-Propylene glycol | | | | | | | | |
| Hesperetin 2,5% in 1,2-Propylene | | | | | 0.1 | | 0.2 | |
| glycol | | | | | | | | |
| Phloretin 2,5% in 1,2-Propylene | | | | 0.2 | 0.2 | | | 0.2 |
| glycol | | | | | | | | |

| | |
|---|---|
| Water | ad 100 |

The ingredients are mixed in the stated sequence and the finished ketchup is homogenized using an agitator, poured into bottles and sterilized.

### APPLICATION EXAMPLE 7

### Reduced-Sugar Fruit Gums

| | | B ((% by weight), |
|---|---|---|
| | A (% by weight), | preparation |
| | Comparative | according to the |
| Ingredient | preparation | present invention |
| Water | 23.70 | 25.60 |
| Saccharose | 34.50 | 8.20 |
| Glucose syrup, DE 40 | 31.89 | 30.09 |
| Iso Syrup C* Tru Sweet 01750 (Cerestar GmbH) | 1.50 | 2.10 |
| Gelatin 240 Bloom | 8.20 | 9.40 |
| Polydextrose (Litesse ® Ultra, Danisco Cultor | — | 24.40 |
| GmbH) | | |
| Yellow and red colourings | 0.01 | 0.01 |
| Citric acid | 0.20 | |
| Cherry flavouring, containing 1% by weight of | — | 0.30 |
| 6″-O-acetyl- trilobatin, compound (1), based on | | |
| the flavouring | | |

Note: Polydextrose is itself a non-sweet-tasting polysaccharide with a low calorific value.

### APPLICATION EXAMPLE 8

### Carbonated Drink (Flavour Direction: Cola)

A: drink containing sugar (comparative) drink
B: low-calorie drink
C: low-calorie drink
D: low-calorie drink
E: low-calorie drink

| | A | B | C | D | E |
|---|---|---|---|---|---|
| | (% by | (% by | (% by | (% by | (% by |
| Ingredient | weight) | weight) | weight) | weight) | weight) |
| Phosphoric acid 85% | 0.635 | 0.635 | 0.635 | 0.635 | 0.635 |
| Citric acid, anhydrous | 0.064 | 0.064 | 0.064 | 0.064 | 0.064 |
| Caffeine | 0.064 | 0.064 | 0.064 | 0.064 | 0.064 |
| Sucrose | 63.60 | — | — | — | 12.9 |
| Sucralose | — | 0.126 | — | — | — |
| Erythritol | — | — | 6.000 | — | — |
| Aspartame | — | — | 0.350 | — | — |
| Rebaudioside A | — | — | — | 0.300 | 0.100 |
| Rebaudioside M | — | — | — | — | 0.200 |
| Acesulfame K | — | — | 0.07 | — | — |
| Sugar colouring | 0.800 | 0.800 | 0.800 | 0.800 | 0.800 |
| Cola type drink emulsion | 1.445 | 1.445 | 1.445 | 1.445 | 1.445 |
| Sodium benzoate | 0.106 | 0.106 | 0.106 | 0.106 | 0.106 |
| 6″-O-Acetyltrilobatin, compound | — | 2.0 | 2.0 | 2.0 | 2.0 |
| (1), 0.5% in 1,2-propylene glycol | | | | | |

| | |
|---|---|
| Water | ad 100 |

The solid components or ingredients are individually mixed with water, combined and made up to 100 g with water. The concentrate obtained is then allowed to age over night at ambient temperature. Finally, 1 part concentrate is mixed with 5 parts carbonated water, filled into bottles and sealed.

### APPLICATION EXAMPLE 9

### Drink Chocolate Instant Powder

Preparation A: standard preparation
Preparation B-D: preparation according to the present invention

| Ingredient | Use in weight % | | | |
|---|---|---|---|---|
| Preparation | A | B | C | D |
| Sucrose, extra fine | 73.5 | 48.475 | 73.69 | 13.325 |
| Cocoa powder alkalized, | 25.0 | 25.0 | 25.0 | 25.0 |
| 10-12% by weight fat | | | | |
| Maltodextrin DE15-19 | 0.43 | 0.43 | 0.43 | 0.43 |
| from corn starch | | | | |
| Salt (NaCl), extra fine | 0.43 | 0.43 | 0.43 | 0.43 |
| Ascorbic acid | 0.29 | 0.29 | 0.29 | 0.29 |
| Vitamin mix M8 40-1507 | 0.145 | 0.145 | 0.145 | 0.145 |
| D-Allulose, crystalline | — | 25 | — | 60 |
| 6″-O-Acetyltrilobatin, | — | 0.01 | 0.01 | 0.01 |
| compound (1) | | | | |
| Hesperetin | — | — | 0.01 | 0.01 |
| dihydrochalcone (I) | | | | |
| Hesperetin | — | 0.05 | — | — |
| Rebaudioside A | — | — | — | 0.02 |
| Phloretin | — | 0.03 | — | — |
| Rebaudioside M | — | — | — | 0.30 |
| Phyllodulcin (70%, dried | — | 0.01 | — | 0.01 |
| Oamacha extract) | | | | |

Standard dosage in milk for preparing an choco beverage: 6.9% by weight.

### APPLICATION EXAMPLE 10

### Instant Ice Tea Type Peach

Preparation A: standard preparation
Preparation B-D: preparation according to the present invention

| Ingredient | Use in weight % | | | |
|---|---|---|---|---|
| Preparation | A | B | C | D |
| Sucrose, extra fine | 95.06 | 70.09 | 70.25 | — |
| Citric acid, | 2.2 | 2.0 | 2.0 | 1.8 |
| anhydrous | | | | |
| Black Tea extract, | 1.34 | 1.34 | 1.34 | 1.34 |
| powdered | | | | |
| Peach aroma spray | 0.66 | 0.66 | 0.66 | 0.66 |
| dried | | | | |
| Tea aroma type | 0.27 | 0.27 | 0.27 | 0.27 |
| Ceylon dried | | | | |
| Trisodium citrate | 0.25 | 0.25 | 0.25 | 0.25 |
| Ascorbic acid | 0.22 | 0.22 | 0.22 | 0.22 |
| Polydextrose | — | — | — | 25 |
| D-Allulose, | — | 25 | 25 | 70 |
| crystalline | | | | |
| 6″-O-Acetyltrilobatin, | — | 0.02 | 0.02 | 0.01 |
| compound (1) | | | | |
| Phyllodulcin (80%, | — | — | 0.01 | 0.01 |
| dried Oamacha | | | | |
| extract) | | | | |
| Hesperetin | — | 0.05 | — | — |
| Rebaudioside A | — | — | — | 0.01 |
| Phloretin | — | 0.02 | — | — |
| Rebaudioside M | — | — | — | 0.20 |
| Homoeriodictyol- | — | 0.1 | — | 0.05 |
| Sodium salt | | | | |

Standard dosage in water for preparing a peach ice tea beverage: 7.5% by weight.

### APPLICATION EXAMPLE 10

### Comparative Example with Sucrose (A)

Preparation (wt.-% or ppm if specified accordingly)

| Ingrediens | A | B | C | D | E | F | G | H |
|---|---|---|---|---|---|---|---|---|
| sucrose | 7 | 3.5 | 3.5 | 3.5 | 3.5 | 3.5 | 1.5 | — |
| D-allulose | — | 2 | — | 2 | — | 2 | — | 7 |
| 6″-O-Acetyltrilobatin, | — | 30 ppm | 10 ppm | 10 ppm | 10 ppm | 20 ppm | 5 ppm | 10 ppm |
| compound (1) | | | | | | | | |
| Hesperetin (HT) | — | — | 5 ppm | — | — | — | — | 5 ppm |
| Phloretin (PH) | — | — | — | 15 ppm | — | — | — | — |
| Hesperetin | — | — | — | — | 15 ppm | — | 5 ppm | — |
| dihydrochalcone (HC) | | | | | | | | |
| Alpha-glycosylated | — | — | — | — | — | 10 ppm | 10 ppm | — |
| rubusosides (OR) | | | | | | | | |
| Phyllodulcin (75%, as | — | — | — | — | — | — | — | 5 ppm |
| dried Oamacha extract) | | | | | | | | |
| Citric acid | 0.15 | 0.15 | 0.15 | 0.15 | 0.15 | 0.15 | 0.15 | 0.15 |
| ascorbic acid | 0.02 | 0.02 | 0.02 | 0.02 | 0.02 | 0.02 | 0.02 | 0.02 |
| Black tea extrakt | 0.15 | 0.15 | 0.15 | 0.15 | 0.15 | 0.15 | 0.15 | 0.15 |
| natural lemon flavor | 0.1 | 0.1 | 0.1 | 0.1 | 0.1 | 0.1 | 0.1 | 0.1 |

| | |
|---|---|
| water | to 100% |

The ingredients were mixed in the order listed in the order listed into bottled and sterilized.

The invention has been made in the context of a partially publicly sponsored project (sponsor BMBF, FKZ 13GW0226C and 13GW0226B).

1-12. (canceled)

13. A composition comprising:
(a) a compound of formula (I), 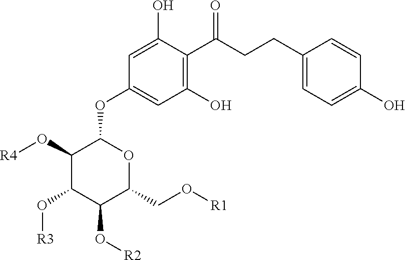
wherein, R1 is acetyl and R2, R3, and R4 are hydrogen;
(b) one or more sweet tasting substances; and
(c) optionally, one or more sweet taste modulating substances.

14. The composition of claim 13, wherein the compound of formula (I) is obtained by enzymatic alkanoylation of a plant extract.

15. The composition of claim 13, wherein the one or more sweet tasting substances of (b) are natural occurring sweet tasting substances selected from are selected from sweet tasting carbohydrates, sugar alcohols, D-amino acids and salts thereof, steviolgylcoside, stevioside, mono-, di-, tri- or tetra-alpha-glycosylated steviosides or rebaudiosides, steviolbiosid, rebaudiosides, rebaudioside A, rebaudioside B, rebaudioside C, rebaudioside D, rebaudioside E, rebaudioside F, rebaudioside G, rebaudioside H, rebaudioside M, rebaudioside N, rebaudioside X, dulcoside, rubusoside, mono-, di-, tri- or tetra-alpha-glycosylated rubusosides, suavioside A, suavioside B, suavioside G, suavioside H, suavioside I, suavioside J, baiyunoside 1, baiyunoside 2, phlomisoside 1, phlomisoside 2, phlomisoside 3, phlomisoside 4, abrusoside A, abrusoside B, abrusoside C, abrusoside D, cyclocaryoside A, cyclocaryoside I, oslandin, polypodoside A, strogin 1, strogin, 2, strogin 4, selligueanin A, dihydroquercetin-3-acetate, perillartin, telosmosid A15, periandrin I-V, pterocaryoside, cyclocaryoside, mukurozioside, trans-anethol, bryoside, bryonoside, bryonodulcoside, camosifloside, scandenoside, gypenoside, hematoxylin, cyanin, chlorogenic acid, albiziasaponin, telosmoside, gaudichaudiosid, balansin A, balansin B, mogrosides, hernandulcine, monatin, glycyrrhetinic acid and its derivatives, extracts of Thaumatococcus or Stevia ssp., stevia leaf extract, swingle extracts, extracts of Glycerrhyzia ssp., extracts of Rubus ssp., extracts of Mycetia balansae, synthetic sweet tasting substances, or mixtures thereof.

16. The composition of claim 13, wherein the one or more sweet tasting substances of (b) are selected from sucrose, fructose, glucose, steviosides, rebaudiosides, rebaudioside A, rebaudioside M, mono-, di-, tri- or tetra-alpha-glycosylated steviosides or rebaudiosides, rubusoside, mono-, di-, tri- or tetra-alpha-glycosylated rubusosides.

17. The composition of claim 13 comprising (iii).

18. The composition of claim 17, wherein the one or more sweet-taste modulating substances of (iii) are selected from hesperetin, hesperetin dihydrochalcone, naringenin, phloretin, eriodictyol, homoeriodictyol, phyllodulcin, neohesperdindihydrochalkon, naringindihydrochalkon, phloretin, extracts of Hydrangea macrophylla ssp. serrata, Amacha or Amagi amacha comprising active amounts of phyllodulcin, or mixtures thereof.

19. The composition of claim 13, wherein the composition is a product and the compound of formula (I) is in an amount of less than 150 ppm, based on the total weight of the product.

20. The composition of claim 13, wherein the composition is a pharmaceutical product for oral application, a product for oral care, a product for nutrition, or a product for pleasure.

21. A method for optimizing flavor of one or more sweet tasting substances comprising:
(i) providing a compound of formula (I), 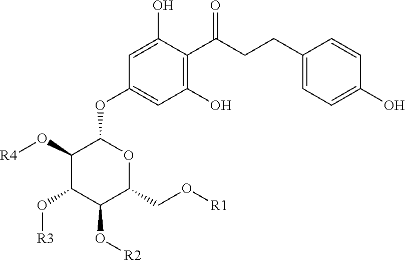
wherein, R1 is acetyl and R2, R3, and R4 are hydrogen, and the compound is obtainable by enzymatic alkonoylation of a plant extract;
(ii) combining the compound of formula (I) with one or more sweet tasting substances of (i); and
(iii) optionally, combining the compound of formula (I), the one or more sweet tasting substances of (i), or mixture thereof, with one or more sweet-taste modulating substances.

22. The method of claim 21, wherein the method increases sweet flavor, improves mouthfeel, reduces acidity, reduces bitterness, or reduces off-taste of the one or more sweet tasting substances of (ii).

23. The method of claim 21, wherein the compound of formula (I) is obtained by enzymatic alkanoylation of a plant extract.

24. The method of claim 21, wherein the one or more sweet tasting substances of (b) are naturally occurring sweet tasting substances selected from sweet tasting carbohydrates, sugar alcohols, D-amino acids and salts thereof, steviolgylcoside, stevioside, mono-, di-, tri- or tetra-alpha-glycosylated steviosides or rebaudiosides, steviolbiosid, rebaudiosides, rebaudioside A, rebaudioside B, rebaudioside C, rebaudioside D, rebaudioside E, rebaudioside F, rebaudioside G, rebaudioside H, rebaudioside M, rebaudioside N, rebaudioside X, dulcoside, rubusoside, mono-, di-, tri- or tetra-alpha-glycosylated rubusosides, suavioside A, suavioside B, suavioside G, suavioside H, suavioside I, suavioside J, baiyunoside 1, baiyunoside 2, phlomisoside 1, phlomisoside 2, phlomisoside 3, phlomisoside 4, abrusoside A, abrusoside B, abrusoside C, abrusoside D, cyclocaryoside A, cyclocaryoside I, oslandin, polypodoside A, strogin 1, strogin, 2, strogin 4, selligueanin A, dihydroquercetin-3-acetate, perillartin, telosmosid A15, periandrin I-V, pterocaryoside, cyclocaryoside, mukurozioside, transanethol, bryoside, bryonoside, bryonodulcoside, carnosifloside, scandenoside, gypenoside, hematoxylin, cyanin, chlorogenic acid, albiziasaponin, telosmoside, gaudichaudiosid, balansin A, balansin B, mogrosides, hernandulcine, monatin, glycyrrhetinic acid and its derivatives, extracts of Thaumatococcus or Stevia ssp., stevia leaf extract, swingle extracts, extracts of Glycerrhyzia ssp., extracts of Rubus ssp., extracts of Mycetia balansae, synthetic sweet tasting substances, or mixtures thereof.

25. The method of claim 21 comprising (iii).

26. The method of claim 25, wherein the one or more sweet-taste modulating substances of (iii) are selected from hesperetin, hesperetin dihydrochalcone, naringenin, phloretin, eriodictyol, homoeriodictyol, phyllodulcin, neohesperdindihydrochalkon, naringindihydrochalkon, phloretin, extracts of Hydrangea macrophylla ssp. serrata, Amacha or Amagi amacha comprising active amounts of phyllodulcin, or mixtures thereof.

27. Method of producing a compound of formula (I) of claim 13 comprising:
(i) providing trilobatin, an extract from Lithocarpus litseifolius or Lithocarpus polystachyus leaf material, an extract from Malus trilobata root material, or mixtures thereof; and
(ii) subjecting the trilobatin or the extract of (i) to a chemical or enzymatic alkanoylation.

28. The method of claim 27, wherein the trilobatin or the extract of (i) is incubated with an acyl donor and a lipase.

29. The method of claim 27, wherein the acyl donor is selected form triacetin, tripropionin, tributyrin, acetic acid, acetic anhydride, ethyl acetate, ethyl propionate, ethyl butanoate, or mixtures thereof.

30. The method of claim 27, wherein the lipase is from a microorganism selected from Candida antarctica A, Candida antarctica B, Candida rugosa, Burkholderia cepacia, Rhizopus sp., Rhizomucor miehei, Mucor javanicus, Yarrowia lypolytica, Geotrichum candidum, Aspergillus niger, Aspergillus oryzae, Pseudomonas alcaligenes, Pseudomonas mendocina, Thermomyces lanuginosus, Chromobacterium viscosum, or mixtures thereof.
